(19) United States
(12) Patent Application Publication
(10) Pub. No.: US 2009/0012561 A1
(43) Pub. Date: Jan. 8, 2009

(54) Tissue Anchor

(75) Inventor: Alan John Johnstone, Aberdeen (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Grampian Health Board; The Robert Gordon University

(21) Appl. No.: 11/667,648

(22) PCT Filed: Nov. 16, 2005
(86) PCT No.: PCT/GB05/04409

§ 371 Date: Oct. 11, 2007

(30) Foreign Application Priority Data
Nov. 17, 2004 (GB) 0425296.1

(51) Int. Cl. A61B 17/04; A61B 17/32

(52) U.S. Cl. 606/232; 606/167

(57) ABSTRACT

The invention provides a tissue anchor comprising a first portion and a second portion. At least one point is provided on the first portion and a resilient device is provided between the first and second portions. The one or more points on the first portion can be sharpened and capable of penetrating dense tissue. The resilient device biases the first and second portions apart from one another in order to urge them into a second open configuration. The first and second portions are movable to a closed delivery configuration. The first and second portions are urged towards one another against the bias of the resilient device in the closed delivery configuration.

The present invention relates to a tissue anchor, particularly for use in bone, cartilage and other tissues of a body.

Tissue anchors are widely used in surgery to retain and anchor sutures or other restraining devices. Present designs generally employ asymmetric anchors that are inserted into drilled holes in a bone. In some cases anchors are provided with threads to engage within a threaded hole, or rely on their asymmetric designs for anchorage within the bone or other tissue. Existing designs of tissue anchors are generally bulky, which limits their usefulness in certain bones, e.g. hands and feet.

According to the invention there is provided a tissue anchor comprising a first portion and a second portion, at least one point on the first portion and a resilient device between the first and second portions.

The point on the first portion is sharpened to penetrate the dense tissue when the first and second portions are held in a first configuration relative to one another. Once the point has penetrated the tissue e.g. bone, the resilient device applies a force between the first and second portions in order to move them into a second configuration that is adapted to resist withdrawal from the tissue in the opposite direction of penetration.

Typically, the tissue anchor has a barb pointing in the opposite direction to the main point on the first portion. In preferred embodiments, the first and second portions are mirror image parts with forward facing points and corresponding barbs that extend in a different direction.

In a preferred format, each portion is generally in the form of a hook with a forward pointing end to penetrate the tissue, sloping back to a rearward pointing barb at the trailing end. The or each barb typically extends radially outward further than the points at the leading end.

The resilient device can simply be a resilient wire or piece of sprung plastics material or metal such as sprung steel connecting the two hooks, and in preferred embodiments, the resilient device biases the two portions apart from one another in the absence of any force.

When the device is to be delivered, the two portions are typically forced toward one another against the natural bias of the resilient device before the tissue anchor is delivered into the tissue. In preferred embodiments of the device with the double hook configuration, this forces the tips of the respective hooks on the first and second portions towards one another so that they meet at their leading edge points, and the force applied typically maintains them in this first configuration while the tissue anchor is being delivered. Once the tissue anchor has been delivered to the desired location in the tissue, the force maintaining the device in the first configuration is removed, and in the preferred embodiments, the resilient member then splays apart the points at the leading ends of the first and second portions, and also moves the barbs further apart in order to lodge the tissue anchor securely within the tissue under the force applied by the resilient device.

Optionally, the tissue anchor can be twisted around its axis of insertion, so as to lodge the barbs and points more firmly within the tissue.

In some embodiments, the tissue anchor is forced into the closed configuration for delivery (with the leading edge points of the hooks forced together as described above) by threading a suture through the tissue anchor and threading the suture through a delivery sleeve, and then pulling the suture and the tissue anchor relative to the delivery sleeve so that the sleeve slides along the suture and applies a force to the tissue anchor. This force applied by the delivery sleeve typically moves the first and second portions from their naturally splayed open configuration to their closed configuration with the leading edge points forced together. The pressure applied via the suture and the delivery sleeve typically maintains the tissue anchor in the closed configuration all the way through the delivery process, and one advantage of this is that the tissue anchor can be hammered into place using the delivery sleeve as an anvil.

In some other embodiments the tips of the hooks are pressed together as outlined above by the action of the resilient device, which can be forced into a suitable configuration to achieve this by the confines of the delivery sleeve. For example, some of the resilient devices according to this embodiment can be generally teardrop shaped in the form of a loop with the hooks connected at the narrowed end. In such embodiments, the loop part of the drop is compressed by the confines of the delivery sleeve, so as to push the tips together. This kind of embodiment avoids the need to pull the suture or to apply other external force to it to keep the tips of the hooks together during insertion.

Once the tissue anchor is in the required position, the delivery sleeve can simply be withdrawn from the tissue, leaving the tissue anchor fixed in the correct position, and the suture already trailing out of the required path.

Typically the diameter of the delivery sleeve is less than the diameter of the barbs of the tissue anchor when in the closed configuration. This allows withdrawal of the delivery sleeve without disturbing the tissue anchor, as the rearward facing barbs can bite into solid bone beyond the nominal diameter of the aperture in the tissue made by the delivery sleeve.

If the tissue anchor is to be withdrawn from the tissue, it can be forced into the closed configuration for withdrawal by insertion of a larger diameter recovery sleeve, either over the delivery sleeve or simply over the suture. The diameter of the recovery sleeve is typically wider than the diameter of the splayed barbs in the second configuration, so that during withdrawal of the tissue anchor from the tissue, the rearward facing barbs no longer impede withdrawal of the tissue anchor.

A leading edge of the recovery sleeve can be provided with cutting formations to cut a hole in the tissue and thereby facilitate removal of the sleeve. An internal surface of the recovery sleeve can be provided with an annular groove to accommodate the laterally outermost barbs on the tissue anchor.

According to the first aspect of the invention, there is also provided a method of setting an anchor in tissue, the method comprising the steps of:
providing a tissue anchor having a first portion and a second portion;
forcing the first and second portions into a first configuration in which the tissue anchor stores energy in a resilient device;
inserting at least one point provided on the first portion into the tissue; and
anchoring the tissue anchor within the tissue by removing the force on the first and second portions to allow the first and second portions to occupy a second configuration in which the first and second portions are spaced relative to one another.

The method can include the step of penetrating dense tissue with the point of the first portion.

The method can also include urging the first and second portions into the first configuration against the bias of the resilient device prior to inserting the tissue anchor into the tissue. The method can include maintaining the first and second portions in the first configuration during insertion of the tissue anchor into the tissue.

The method can include introducing the tissue anchor into a delivery sleeve, which forces the tissue anchor into the second configuration. The method can include withdrawing the delivery sleeve from the tissue anchor and permitting the tissue anchor to change from the first configuration to the second configuration.

According to a second aspect of the invention, there is provided a method of recovering a tissue anchor disposed in a tissue, the tissue anchor having a first portion and a second portion spaced apart from one another into a second configuration by means of a resilient device, whereby the tissue anchor is secured within the tissue, the recovery method comprising the steps of urging the first and second portions into a first configuration in which the first and second portions are urged against the bias of the resilient device, engaging the tissue anchor within a recovery sleeve and recovering the tissue anchor by withdrawing the recovery sleeve.

The method can include introducing the tissue anchor into a sleeve having a diameter less than the lateral extent of hooks provided on each of the first and second portions and urging the sleeve against a rear face of each hook to thereby urge the first and second portions against the bias of the resilient device.

The method can include accommodating a suture coupled to the tissue anchor in a throughbore of the sleeve. The method can also include sliding the sleeve along the anchored suture and thereby guiding the sleeve through the tissue to the tissue anchor to facilitate removal or adjustment thereof.

The method can also include accommodating the sleeve and the tissue anchor within the recovery sleeve before withdrawing the recovery sleeve and recovering the tissue anchor.

The method can also include retaining the tissue anchor within the bore of the recovery sleeve by providing an annular groove on an internal surface of the recovery sleeve and engaging the laterally outermost portion of the hooks in the groove.

The method can further include cutting through tissue using a leading end of the recovery sleeve to access the tissue anchor prior to its recovery.

An embodiment of the present invention will now be described by way of example, and with reference to the accompanying drawings, in which:—

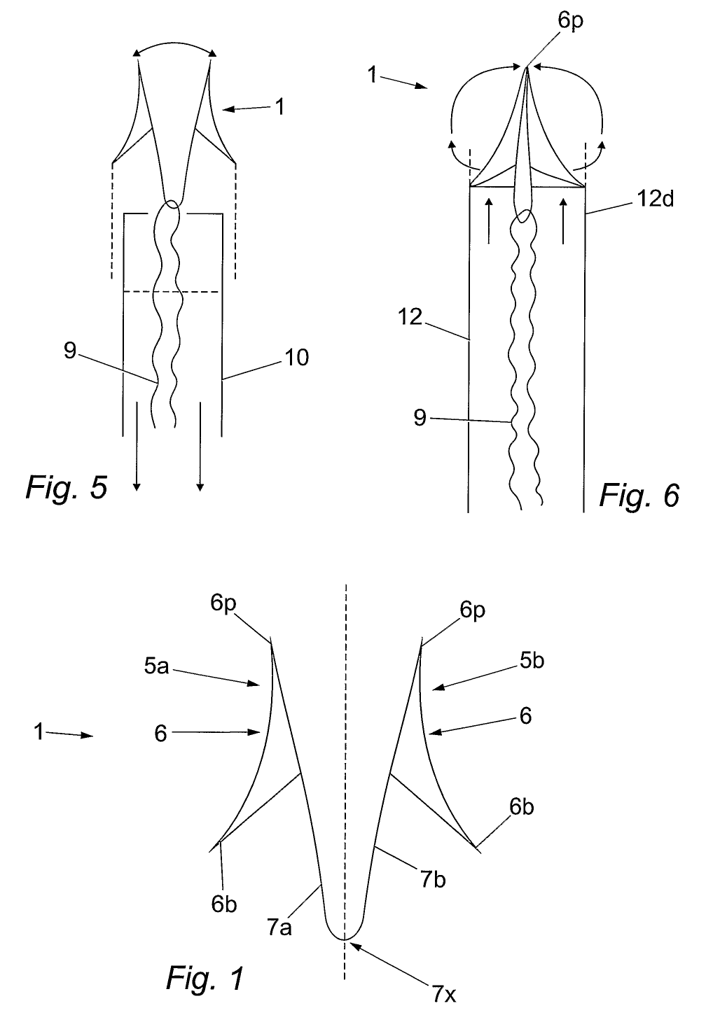

FIG. 1 shows a tissue anchor in an open configuration;

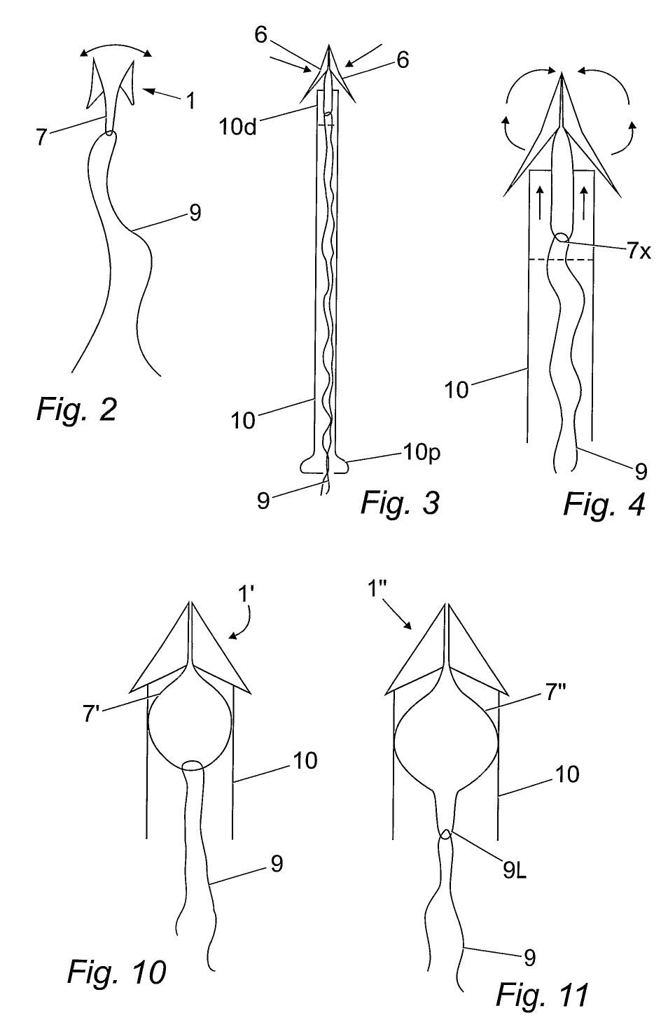

FIG. 2 is a side view of a tissue anchor in an open configuration in which the rearward facing barbs resist withdrawal of the tissue anchor from a body with a suture attached;

FIG. 3 is a side view of the FIG. 1 tissue anchor with a delivery sleeve;

FIG. 4 is a close up view of the FIG. 2 tissue anchor and sleeve in the closed configuration with force applied via the delivery sleeve and suture to close the points of the tissue anchor;

FIG. 5 is a side view similar to FIG. 3, but showing the removal of force applied via the delivery sleeve and suture, and the splaying of the points of the tissue anchor as a result of force applied by the resilient device;

FIG. 6 is a side view similar to FIGS. 2 to 4, showing the tissue anchor being closed by force applied by a suture and a recovery tube;

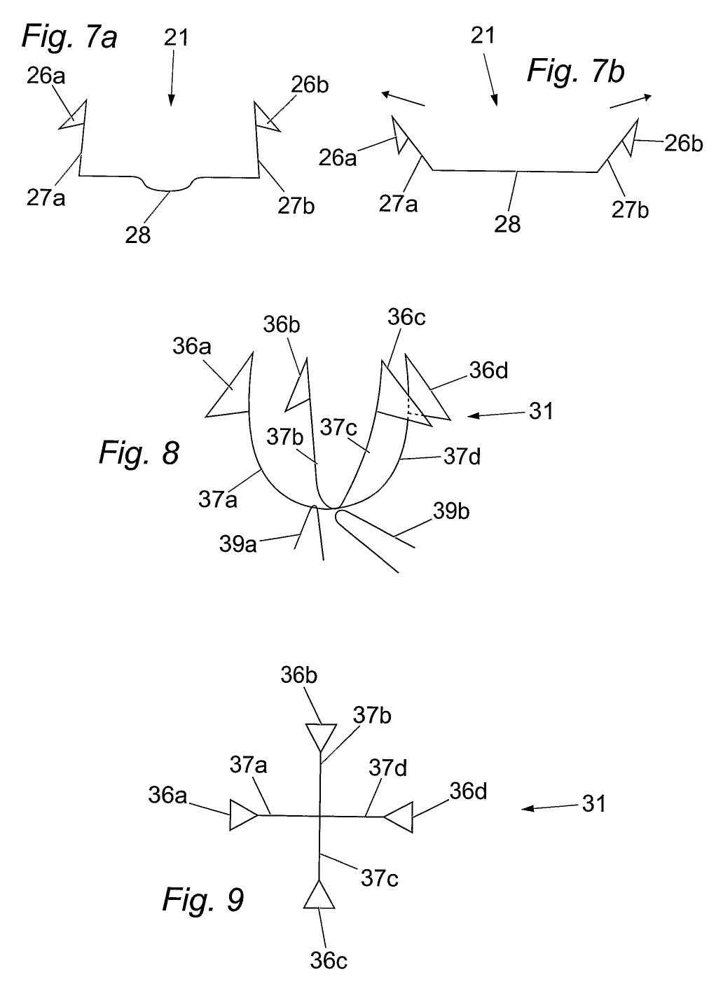

FIGS. 7a and b are side views of a second embodiment of a tissue anchor showing the anchor in its closed configuration for insertion (a) and in the open configuration (b) after withdrawal of the delivery sleeve;

FIG. 8 is a perspective view of a third embodiment of a tissue anchor;

FIG. 9 is a plan view of the FIG. 8 embodiment;

FIG. 10 is a side view of a further embodiment of a tissue anchor; and

FIG. 11 is a side view of a further embodiment of a tissue anchor.

Referring now to the drawings, FIG. 1 shows a side view of a tissue anchor 1 having first and second portions in the form of arms 5a and 5b. Each arm 5 comprises a hook portion 6 having a sharpened point 6p at the leading end of the anchor 1, and a barb 6b pointing away from the leading end. The hook portion 6 is optionally smoothly curved between the barbs 6b and point 6p, so that the tip of the barb 6b is spaced radially outwards from the point 6p. The lower surface 61 of the hook 6 is canted at an angle of less than 90° with respect to the arm, so that the barbs 6b on each hook portion points away from the leading edge of the device (at the point 6p).

Each arm is formed from a stiff wire or steel strip. In the embodiments shown in the drawings, the two arms are made from a single continuous flexible steel strip or wire, but it would be acceptable to form the arms separately, either from a resilient material or a rigid material, and connect them by a resilient device such as a leaf spring, a hinge or some other device.

Referring now to FIG. 2, the arms are connected in a general V-shape with the resilient device at the apex 7x of the V. Other shapes could be used apart from V-shapes. In the embodiment shown, the two arms are formed from a continuous piece of sprung steel, to the ends of which the hooks are attached as shown. The apex 7x of the V serves as the resilient device and the arms are thereby biased apart into the general V-shape shown in FIGS. 1 and 2 in the resting position. The inherent resilience of the sprung steel strip allows the tissue anchor to flex as shown in FIG. 2 when force is applied to it, and returns the tissue anchor to the open configuration shown in FIGS. 1 and 2 when the force is removed.

Each of the hooks 6 has three sides in the embodiments shown. One side extending between the point 6p and the barb 6b is generally arcuate and adopts a slightly concave configuration. The concave configuration assists in the passage of the hook through the tissue, but a straight side would suffice in this embodiment. Another side of the hook 6 connects the hook to the end of the arm 7. The end of the arm 7 is generally resistant to flex, either as a result of the connection of the hook at that point, or by being made of an inherently stiffer material at the end of the arm. The remaining lower side 61 of the hook extends between the barb 6b and the arm 7. The angle made between this lower side 61 of the hook 6 and the arm 7 is generally less than 90°, so that the barb 6b faces away from the leading end at the point 6p, and towards the trailing end at the apex 7x between the arms 7. An angle of 90° in this instance would also suffice, but the acute angle made by the rearward facing lower side 61 of the hook enables the barbs 6b to anchor more effectively within the tissue after insertion, and facilitates loading of the device to push the tips 6p together by loading the delivery sleeve.

In use, the natural position of the tissue anchor 1 is as shown in FIG. 1 and FIG. 2, with the points 6p of the hooks 6 splayed apart by the natural resilience of the sprung steel in the arms 7. A suture 9 is threaded between the arms 7a, 7b as shown in FIG. 2 and the free ends of the suture are then gathered and inserted into a distal end 10d of a delivery tube 10, the free ends of the suture 9 being recovered from the proximal end 10p of the delivery tube 10, as shown in FIG. 3. The suture 9 can then be pulled through the proximal end 10p of the delivery tube 10. This pulls the apex 7x and the arms 7a, 7b down into the delivery tube, and pulls the lower sides 61 of the hooks against the proximal end 10b of the delivery tube 10. This forces the points 6p on each hook together as shown in FIG. 4. In the embodiments shown in FIG. 3 and FIG. 4, the outer diameter of the delivery tube 10 is less than the distance between the barbs 6b in the closed position shown in FIG. 4, so that the ends of the barbs 6b protrude radially beyond the outer diameter of the delivery tube 10, and the outer diameter of the delivery tube 10 presses against the middle of the rear facing lower side 61 of the hook 6.

Once the suture 9 has been pulled tight through the delivery tube 10, and the points 6p of the leading end of the tissue anchor 1 are closed together as shown in FIG. 3 and FIG. 4, the tissue anchor is then inserted into the body while the tension on the suture 9 is maintained, thereby keeping the points 6p of the hook together. The tissue anchor 1 can be inserted either by simply pushing the delivery sleeve into the tissue, or by hammering the proximal end 10p of the delivery sleeve. Once the tissue anchor 1 is in the required position as judged by the angle and depth of insertion of the delivery sleeve into the tissue, the assembly can then optionally be forcibly rotated around the axis of the delivery sleeve 10 to twist the barbs 6b in one or both rotational directions. This twisting movement moves the barbs 6b out of the path that the hook 6 has cut through the tissue during insertion, and tends to lodge the barbs 6b in sound bone or other tissue thereby further resisting withdrawal of the tissue anchor 1 therefrom.

At that point, the tension applied to the suture 9 relative to the delivery sleeve 10 can be removed, allowing removal of the delivery sleeve 10, and leaving the tissue anchor 1 firmly lodged in the tissue. When the tension is removed from the suture 9 and the distal end 10d is withdrawn from the hooks 6, the natural resilience of the resilient device on the tissue anchor 1 splays the arms 7a, 7b apart from one another, and moves the hooks 6, and thus the barbs 6b further apart as shown in FIG. 5, thereby further embedding the tissue anchor within the tissue, and resisting withdrawal. The delivery sleeve 10 can be withdrawn over the suture 9, leaving the suture 9 in place in the path cut by the tissue anchor 1 during insertion. The suture can then be fastened to another tissue anchor, or to other implant devices or fastened to other tissues as necessary.

If the tissue anchor is to be removed, either at completion of treatment, or because of incorrect placement, the delivery sleeve 10 can be reinserted to close the tissue anchor 1 and permit withdrawal, but in preferred embodiments, removal is facilitated by a separate recovery sleeve 12 as shown in FIG. 6. The recovery sleeve 12 typically has a wider outer diameter than the delivery sleeve 10, and when placed over the suture 9 (with or without the delivery sleeve being present) and pushed against the tissue anchor 1, the distal end 12d of the recovery sleeve 12 abuts against the tips of the barb 6b, and the outer diameter of the recovery sleeve 12 is typically larger than the distance between the barbs 6b while in the closed position. Therefore, the rearward facing barbs 6b can be shielded by the recovery sleeve 12 so that they do not impede withdrawal from the tissue when engaged by the recovery sleeve 12. The suture 9 is optionally tensioned relative to the recovery sleeve in the same way as described for the delivery sleeve 10 in order to close the points 6p of the hook 6, so that the tissue anchor 1 can then be withdrawn from the tissue without barbs etc causing damage to the tissue during the withdrawal process. The angle made between the rear facing side of the hook 6 and the arm 7 is typically sufficiently acute so as to create an acute angle in both open and closed configurations of the device. Suitable angles can be 50° to 80°. In some embodiments of the device, the recovery sleeve 12 can have cutting formations such as bevelled or chiselled leading edges on its distal tip to enlarge holes made by the insertion of the anchor. The delivery sleeve may also optionally have an annular groove in the distal end to accommodate the radially outermost edges of the hooks.

FIG. 7 shows a modified embodiment of a tissue anchor 21 in the form of a staple, with hooks 26, arms 27, and a bar 28 connecting the two arms. The bar 28 can be sprung to provide the resilient device, and can take the form of a leaf spring, or simply a section of sprung steel can be provided to form the connection between the arms 27 and the bar 28. The resilient device(s) biases the arms 27 into the open position shown in FIG. 7b, and the tissue anchor 21 can be inserted in the closed configuration shown in FIG. 7a as described for the previous embodiment, by using a delivery sleeve to force the arms 27 into the closed configuration.

FIG. 8 shows a further embodiment of a tissue anchor 31 having hooks 36, and arms 37 as previously described. The embodiment 31 differs from the first embodiment described in that instead of two hooks and two arms it has at least four hooks 36a, b, c, d, each located on a respective arm 37a, b, c, and d. This can be achieved simply by connecting two tissue anchors 1 together by welding the apices together in a cruciform arrangement. It will be appreciated that the tissue anchor 31 shown in FIGS. 8 and 9 can have 3, 4, 5, 6, 7 or any other number of respective arms and hooks, and the example shown is merely illustrative of the principle and is not intended to be limiting. One advantage of the embodiment shown in FIG. 31 is that separate sutures 39a and 39b can be attached to different parts of the arms 37. For example, if the embodiment 31 is considered as a pair of tissue anchors formed from a first tissue anchor comprising arms 37a, 37d and hooks 36a, 36d, and a second tissue anchor comprising arms 37b, 37c and hooks 36b, 36c, wherein the apices of the tissue anchors are connected together by welding, gluing or some other kind of connection then the sutures 39a and b can be looped over any of four different positions, for example over any of the arms 37a, b, c or d. This means that a number of different sutures can be attached to a single tissue anchor 31 without the risk of the sutures interfering with one another. This is extremely useful, because when tension is applied to the suture after the tissue anchor has been lodged within the tissue, it can often happen in prior art devices that the tensioning of one suture causes another suture attached to the same tissue anchor to be trapped by the tensioning of the first suture, which can lead to the surgeon thinking that the second suture has been tied firmly when in fact it is not, and importantly can impede the free running of the second suture, which interferes with knotting techniques. The provision of separate and discreet attachment points provided by the separate arms 37a, b, c and d means that multiple sutures can be attached to the single tissue anchor 31 with a reduced risk of fouling one another during tensioning. The tissue anchor 31 shown here can take up to four separate sutures, one looped over each of arms 37a,b,c and d, but other multiples are clearly within the scope of the invention.

FIG. 10 shows a further embodiment of a tissue anchor 1′, which has arms 7′ formed together and compressed into a general teardrop shape when the device 1′ is in a closed configuration and held within the bore of the delivery sleeve 10. The arms 7′ comprise sprung steel and are kept in the closed configuration by the pressure of the delivery sleeve acting on the loop portion of the arms 7′. As the hooks are connected to the arms at the narrowed end of the teardrop shape, the tips of the hooks are pressed together during insertion into the body by the force exerted on the arms by the inner surface of the bore of the sleeve, and this embodiment requires no additional force to keep it in the closed configuration.

FIG. 11 shows a similar embodiment of a tissue anchor 1″, which also has arms 7″ formed together and compressed into a general teardrop shape when the device 1″ is in a closed configuration and held within the bore of the delivery sleeve 10, similar to the FIG. 10 embodiment. This embodiment 1″ also has a suture loop 91 at the proximal end of the arms 7″ to restrict slippage of the suture around the arms 7″.

The FIGS. 10 and 11 embodiments also place less train on the suture, as they avoid pinching the very thin and fragile suture at the apex of the arms during tensioning.

Modifications and improvements can be incorporated without departing from the scope of the invention. For example, in some configurations, the hooks 6 can be planar as shown in FIGS. 1 to 6, or can be plough-shaped as shown in FIGS. 8 and 9. Other forms of hook can be adopted if desired. In certain embodiments, the insertion sleeve 10 can be provided with graduations (e.g. laser markings) to indicate the depth of insertion in order to assist the surgeon in placing the tissue anchor correctly within the tissue.

1. A tissue anchor comprising a first portion and a second portion, at least one point on the first portion and a resilient device between the first and second portions.

2. A tissue anchor according to claim 1, wherein the at least one point on the first portion is sharpened and capable of penetrating dense tissue.

3. A tissue anchor according to claim 1, wherein the first and second portions are movable to a closed delivery configuration wherein the first and second portions are urged towards one another against the bias of the resilient device.

4. A tissue anchor according to claim 1, wherein the resilient device biases the first and second portions apart from one another in order to urge them into a second open configuration.

5. A tissue anchor according to claim 4, wherein the device is adapted to resist withdrawal from the tissue in the second open configuration.

6. A tissue anchor according to claim 1, wherein the tissue anchor has at least one barb associated with the at least one point and pointing in a different direction thereto.

7. A tissue anchor according to claim 6, wherein the at least one barb and the at least one anchor are arranged to point in substantially opposite directions in the second open configuration.

8. A tissue anchor according to claim 1, wherein the first and second portions are mirror image parts.

9. A tissue anchor according to claim 1, wherein the first and second portions each have at least one point and a corresponding barb.

10. A tissue anchor according to any of claim 6, wherein each of the first and second portions is in the form of a hook with a leading end having a point for penetrating tissue, which point extends back to a rearward pointing barb at the trailing end.

11. A tissue anchor according to any of claim 6, wherein the at least one barb has a greater lateral extent than the at least one point at the leading end.

12. A tissue anchor according to claim 1, wherein the resilient device is a resilient wire.

13. A tissue anchor according to claim 1, wherein the resilient device is a piece of sprung plastics material.

14. A tissue anchor according to claim 1, wherein the tissue anchor is capable of at least a degree of rotational movement around an axis of insertion, so as to lodge the at least one barb and the at least one point within the tissue.

15. A tissue anchor according to claim 1, wherein the resilient device is substantially teardrop shaped in the form of a loop.

16. A tissue anchor system comprising a tissue anchor according to claim 1, and a delivery sleeve adapted to bias the anchor into the closed delivery configuration.

17. A tissue anchor system according to claim 16, wherein the diameter of the delivery sleeve is less than the lateral extent of the barbs of the tissue anchor when in the closed delivery configuration.

18. A tissue anchor system according to claim 16, wherein the delivery sleeve has a throughbore for accommodating a suture, which is coupled to the anchor, within the throughbore.

19. A tissue anchor system comprising a tissue anchor according to claim 1, and a recovery sleeve adapted to accommodate at least a portion of the resilient device.

20. A tissue anchor system according to claim 19, wherein the recovery sleeve is provided of larger diameter than the lateral extent of the barbs of the tissue anchor.

21. A tissue anchor system according to claim 19, wherein a leading edge of the recovery sleeve is provided with cutting formations.

22. A tissue anchor system according to claim 19, wherein an internal surface of the recovery sleeve is provided with an annular groove to accommodate the laterally outermost barbs on the tissue anchor.

23. A tissue anchor system according to claim 19, wherein the recovery sleeve is provided with a throughbore for accommodating a delivery sleeve therein.

24. A method of setting an anchor in tissue, the method comprising the steps of:
providing a tissue anchor having a first portion and a second portion;
forcing the first and second portions into a first configuration in which the tissue anchor stores energy in a resilient device;
inserting at least one point provided on the first portion into the tissue; and
anchoring the tissue anchor within the tissue by removing the force on the first and second portions to allow the first and second portions to occupy a second configuration in which the first and second portions are spaced relative to one another.

25. A method according to claim 24, including the step of penetrating dense tissue with the point of the first portion.

26. A method according to claim 24, including urging the first and second portions into the first configuration against the bias of the resilient device prior to inserting the tissue anchor into the tissue.

27. A method according to claim 26, including maintaining the first and second portions in the first configuration during insertion of the tissue anchor into the tissue.

28. A method according to claim 24, including introducing the tissue anchor into a delivery sleeve before insertion into the tissue, which forces the tissue anchor into the second configuration.

29. A method according to claim 28, including withdrawing the delivery sleeve from the tissue anchor after insertion of the anchor into the tissue, and permitting the tissue anchor to change from the first configuration to the second configuration.

30. A method of recovering a tissue anchor disposed in a tissue, the tissue anchor having a first portion and a second portion spaced apart from one another into a second configuration by means of a resilient device, whereby the tissue anchor is secured within the tissue, the recovery method comprising the steps of urging the first and second portions into a first configuration in which the first and second portions are urged against the bias of the resilient device, engaging the tissue anchor within a recovery sleeve and recovering the tissue anchor by withdrawing the recovery sleeve.

31. A method according to claim 30, including introducing the tissue anchor into a sleeve having a diameter less than the lateral extent of hooks provided on each of the first and second portions and urging the sleeve against a rear face of each hook to thereby urge the first and second portions against the bias of the resilient device.

32. A method according to claim 31, including accommodating a suture coupled to the tissue anchor in a throughbore of the sleeve.

33. A method according to claim 32, including sliding the sleeve along the anchored suture and thereby guiding the sleeve through the tissue to the tissue anchor to facilitate manipulation thereof.

34. A method according to claim 31, including accommodating the sleeve and the tissue anchor within the recovery sleeve before withdrawing the recovery sleeve and recovering the tissue anchor.

35. A method according to claim 30, including retaining the tissue anchor within the bore of the recovery sleeve by providing an annular groove on an internal surface of the recovery sleeve and engaging the laterally outermost portion of the hooks in the groove.

36. A method according to claim 30, including cutting through tissue using a leading end of the recovery sleeve to access the tissue anchor prior to its recovery.
